(19) United States
(12) Patent Application Publication
(10) Pub. No.: US 2026/0043094 A1
(43) Pub. Date: Feb. 12, 2026

(54) COMPOSITION FOR DETECTING TARGET NUCLEIC ACID AND USE THEREOF

(71) Applicant: THE INDUSTRY & ACADEMIC COOPERATION IN CHUNGNAM NATIONAL UNIVERSITY (IAC), Yuseong-gu, Daejeon (KR)

(72) Inventors: Kyung-Ho LEE, Yuseong-gu, Daejeon (KR); Dong-Myung KIM, Seo-gu, Daejeon (KR)

(73) Assignee: THE INDUSTRY & ACADEMIC COOPERATION IN CHUNGNAM NATIONAL UNIVERSITY (IAC), Yuseong-gu, Daejeon (KR)

(21) Appl. No.: 18/870,531

(22) PCT Filed: May 31, 2023
(86) PCT No.: PCT/KR2023/007421

§ 371 Date: Nov. 29, 2024

(30) Foreign Application Priority Data
May 31, 2022 (KR) 10-2022-0066757

(51) Int. Cl. C12Q 1/70; C12Q 1/686; C12Q 1/6897

(52) U.S. Cl. CPC C12Q 1/70; C12Q 1/686; C12Q 1/6897

(57) ABSTRACT

The present invention relates to a composition for detecting a target nucleic acid sequence and a method of detecting a nucleic acid using the same.

### CROSS-REFERENCE TO RELATED APPLICATIONS

This application is a National Phase application under 35 U.S.C § 371 of International Application PCT/KR2023/007421, filed May 31, 2023, which claims the benefit of Korean Patent Application No. 10-2022-0066757, filed May 31, 2022, the entireties of which are incorporated herein by reference.

### INCORPORATION OF SEQUENCE LISTING

The sequence listing that is contained in the file named “OPA23178USseqlist.xml”, which is 62,916 bytes (as measured in Microsoft Windows®) and was created on Oct. 1, 2025, is filed herewith by electronic submission and is incorporated by reference herein.

### TECHNICAL FIELD

The present invention relates to a composition for detecting a target nucleic acid and a method of detecting the nucleic acid using the same.

### BACKGROUND ART

The basis of modern molecular diagnostics is the precise detection of nucleic acids. Most current nucleic acid testing methods involve the use of biological components in an in vitro environment. For example, one of the most widely used technologies, polymerase chain reaction (PCR)-based testing, relies on the amplification of target nucleic acids using DNA polymerase of thermophilic microorganisms. However, since the general PCR reaction amplifies a target genetic material by controlling the reaction temperature dozens of times or more, it has a disadvantage of requiring expensive instruments that can stably implement high and low temperatures in a short period of time.

As an alternative to PCR, isothermal amplification methods have been studied. Isothermal amplification methods allow the amplification of a target genetic material at a single temperature (at room temperature or a high temperature of 65° C. or lower) without changing the reaction temperature, and therefore, expensive instruments for thermal cycling are not required. Because of this advantage, isothermal amplification methods may be easily applied to on-site diagnosis which is difficult to implement with general PCR.

An example of isothermal amplification methods is a loop-mediated isothermal amplification (LAMP) reaction. The LAMP reaction is a reaction that amplifies DNA through a chain displacement reaction by creating a loop structure (stem-loop DNA) at the primer binding site from four primer combinations prepared using six regions selected from a target DNA strand, and allows for amplification by binding to the target ssDNA at an isothermal temperature of 60° C. to 65° C. (Nucleic Acids Res. 2000 Jun. 15; 28(12): e63). However, the LAMP reaction has limitations in that it cannot work on short targets and can only detect nucleic acids consisting of several hundred bases. In addition, the detection of nucleic acids amplified through PCR or isothermal amplification is limited to methods of using double-stranded DNA binding dyes such as SYBR Green or methods of using probe sequences labeled with fluorescent dyes such as TaqMan technology, etc. On the other hand, when a method is developed, which detects an amplified transcription or translation product resulting from transcription and translation reactions of a target nucleic acid instead of amplifying the target nucleic acid itself, detection is possible in various ways through the biological activity of the transcription or translation product, unlike existing methods that have limitations in their detection methods. For example, when transcription and translation reactions are induced so that an enzyme protein is produced by the target nucleic acid, detection thereof is possible by using substrates that exhibit color or fluorescence through conversion by the enzyme, and analysis by fluorescence emitted by the protein itself, such as green fluorescence protein (GFP), etc. is also possible. Therefore, rather than detecting the target nucleic acid amplified by PCR or isothermal amplification methods as traditionally used, when a technology capable of recognizing the target nucleic acid and expressing an active protein therefrom is developed, it is possible to more easily and precisely perform nucleic acid detection.

### DISCLOSURE

### Technical Problem

Under this background, the present inventors have made intensive efforts to develop a novel nucleic acid detection method, and as a result, they found that when a construct is developed and used, in which a target binding region annealing to a target sequence is connected to the upstream of a single strand of a promoter, and a reporter nucleic acid or a reporter protein coding sequence is connected to the downstream thereof, signal generation through expression of the reporter nucleic acid or reporter protein is possible only in the presence of the target nucleic acid, thereby completing the present invention.

### Technical Solution

An object of the present invention is to provide a composition for detecting a target nucleic acid, the composition comprising a sensor DNA.

Another object of the present invention is to provide a method of detecting a target nucleic acid, the method comprising the steps of adding a sample to the composition; and measuring an expression level of a reporter gene by carrying out one or more reactions of cell-free replication, transcription, and protein synthesis.

### Advantageous Effects

When the composition of the present invention for detecting a target nucleic acid is used, nucleic acids in a sample may be simply detected, and accordingly, the composition may be usefully applied in various fields comprising molecular diagnosis.

### BRIEF DESCRIPTION OF THE DRAWING

FIG. 1A shows a process of preparing a sensor DNA using a PCR product comprising uracil and a USER reaction, and FIG. 1B shows a process of preparing a sensor DNA using a restriction enzyme;

FIG. 2 shows the result of verifying that a target binding region of a sensor DNA construct of the present invention binds to complementary ssDNA to generate a Broccoli fluorescent aptamer through cell-free transcription (in vitro transcription);

FIG. 3 shows the results of verifying whether the sensor DNA construct of the present invention anneals to some complementary sequences to express reporter proteins, wherein the target sequences binding with the sensor DNA are marked by T7P (OO/ΞΞ), where the front number indicates the number of nucleotides annealing to the target binding region, among the nucleotides constituting the target sequence, and the back number indicates the number of nucleotides annealing to a single-stranded T7 promoter, among the nucleotides constituting the target sequence;

FIG. 4 shows the results of verifying whether the sensor DNA expresses the reporter protein by changing conditions required for protein synthesis, wherein FIG. 4A shows the result of cell-free transcription from sensor DNA bound to the target sequence, and the experiment of FIG. 4B shows the result of synthesizing sfGFP protein from sensor DNA bound to the target sequence using a PURE system consisting of a purified protein synthesis system, in which the three lanes in the left of FIG. 4A are the results of using the target sequence that overlaps and anneals with the target binding region and T7 promoter sequence of sensor DNA, and the three lanes in the right are the results of using the target sequence that anneals only with the target binding region;

FIG. 5 shows whether the target sequence is detected in a cell-free protein synthesis system using an E. coli crude extract (FIG. 5A) or a PURE system to which Pol I and dNTP were added (FIG. 5B), when the target sequence having a part complementary to the upstream of T7 promoter is not completely annealed with the sensor DNA and has a 5′- or 3′-flap;

FIG. 6 shows Signal-to-Noise (S/N) measured in the presence of 10 nM of target ssDNA in an E. coli crude extract-based cell-free protein synthesis system or in a PURE system to which Pol I and dNTP were added, when sfGFP was used as a reporter protein;

FIG. 7 shows the results of measuring proteins expressed by annealing various concentrations of target ssDNAs with the target binding site of the sensor DNA, wherein FIGS. 7A and 7B show Signal-to-Noise (S/N) ratio of sfGFP according to the DNA concentrations, which is expressed from the original double strand as a positive control (FIG. 7A) or expressed through annealing of the target sequence and the sensor DNA (FIG. 7B), FIGS. 7C and 7D show the total protein expression levels of sfGFP, FLuc, and NLuc and the soluble protein expression order expressed in the E. coli crude extract-based cell-free protein synthesis system (FIG. 7C) and Signal-to-Noise (S/N) ratio of the measured fluorescence (sfGFP) and luminescence (FLuc, NLuc) (FIG. 7D), and FIGS. 7E and 7F show Signal-to-Noise (S/N) ratio according to DNA concentrations by measuring luminescence in the presence of various concentrations of the original double strand as a positive control (FIG. 7E) or the target sequence (FIG. 7F) using NLuc as a reporter protein;

FIG. 8 shows the detection of a base sequence of Parvovirus B19 (PV) using NLuc as a reporter protein, in which some of the base sequence of 1934-1983 of the PV genome were used as target sequences;

FIG. 9 shows the results of detecting DNA of Parvovirus (PV) B19 using NLuc as a reporter protein, in which the target binding region of the sensor DNA was designed to consist of 14 base sequences complementary to a base sequence of 1958-1971 of the PV genome;

FIG. 10 illustrates a method of detecting a nucleic acid according to an embodiment of the present invention; and

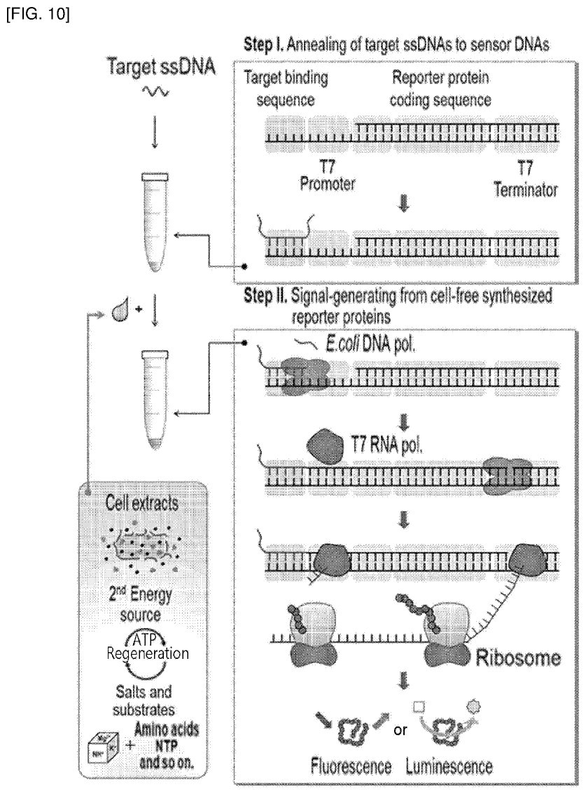

FIG. 11 illustrates a construct comprised in the sensor DNA according to an embodiment of the present invention.

### DETAILED DESCRIPTION OF PREFERRED EMBODIMENTS

The present invention will be described in detail as follows. Meanwhile, each description and embodiment disclosed in this disclosure may also be applied to other descriptions and embodiments. That is, all combinations of various elements disclosed in this disclosure fall within the scope of the present invention. Further, the scope of the present invention is not limited by the specific description described below.

Further, those skilled in the art will recognize, or be able to ascertain using no more than routine experimentation, many equivalents to the specific embodiments of the disclosure described herein. Further, these equivalents should be interpreted to fall within the present invention.

An aspect of the present invention provides a composition for detecting a target nucleic acid, the composition comprising a sensor DNA.

As used herein, the “sensor DNA” refers to a nucleic acid construct comprising a target binding region capable of recognizing a target nucleic acid, a promoter, and a reporter gene linked downstream of the promoter, in which the nucleic acid construct selectively enables expression of the reporter gene only in the presence of the target nucleic acid. The “sensor DNA” of the present invention may be used interchangeably with the term “target-assisted synthesis of reporter (TASR)” or “target-assisted synthesis of enzyme reporter (TASER).”

The terms “TASR and TASER” may also be used interchangeably even when the reporter gene encodes a reporter nucleic acid, such as a Broccoli aptamer or Spinach aptamer, etc., rather than a protein.

Specifically, the sensor DNA of the present invention may comprise the following structure:
(5′) Target nucleic acid sequence-binding region-Promoter-Reporter gene (3′)

In the sensor DNA of the present invention, the target nucleic acid sequence-binding region may be a single-stranded DNA (ssDNA) capable of annealing to the target nucleic acid sequence. Specifically, the single-stranded DNA capable of annealing to the target nucleic acid sequence may comprise a base sequence complementary to all or a part of the target nucleic acid sequence. Specifically, the single-stranded DNA capable of annealing to the target nucleic acid sequence may comprise 4 or more bases. More specifically, the single-stranded DNA capable of annealing to the target nucleic acid sequence may comprise 12 or more bases. For example, when the single-stranded DNA capable of annealing to the target nucleic acid sequence is composed of 4 bases to 11 bases, the target nucleic acid sequence may comprise a promoter sequence. However, the length of the single-stranded DNA is not particularly limited thereto, as long as the reporter nucleic acid or reporter protein may be selectively expressed only in the presence of the target nucleic acid sequence.

In the sensor DNA of the present invention, the promoter may be composed of a single strand.

In one embodiment, the reporter gene downstream of the promoter region comprised in the sensor DNA of the present invention may be in a double-stranded form.

More specifically, the sensor DNA of the present invention may comprise the following structure:
(5′) Target nucleic acid sequence-binding region-T7 promoter-Reporter gene (3′)

The “T7 promoter” of the present invention is a promoter derived from T7 bacteriophage, and the T7 promoter sequence comprised in the sensor DNA of the present invention may be 5′-TAATACGACTCACTATA-3′ or a complementary sequence thereto. However, the present invention is not limited thereto, and other promoter sequences and complementary sequences thereto, or sequences having the same function by addition, deletion, or substitution of one or more bases may be used.

In one embodiment of the present invention, the sensor DNA has a single-stranded T7 promoter, and thus transcription is not initiated by T7 RNA polymerase. Therefore, in an environment without target nucleic acids, the reporter nucleic acid or reporter protein is not expressed. However, in the presence of the target nucleic acids, the target nucleic acid binds to the target nucleic acid sequence-binding region, and then duplexing of the T7 promoter downstream thereof occurs through an extension reaction, thereby allowing the reporter nucleic acid or protein downstream of the T7 promoter region to be expressed.

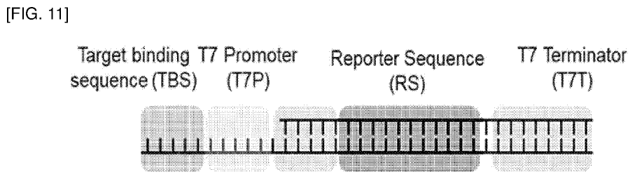

More specifically, the sensor DNA of the present invention may comprise a structure disclosed in FIG. 11, but is not limited thereto.

In one embodiment of the present invention, some portion of the sensor DNA exists as a single strand and some as a double strand, and in order to prepare the sensor DNA, a uracil-specific cleavage reaction may be used, or a restriction enzyme and ligase may be used.

As used herein, the “reporter nucleic acid” refers to a DNA or RNA sequence that is replicated or transcribed from the sensor DNA to generate a signal capable of easily identifying its production or synthesis.

Specifically, the reporter nucleic acid may be an aptamer. For example, a labeling substance may be attached to the aptamer, and for example, the labeling substance may be a fluorescent substance, a radioisotope, a luminescent element, an enzyme, or a nanoparticle, but is not limited thereto.

Specifically, the reporter nucleic acid may be selected from the group consisting of a malachite green aptamer binding with a malachite green dye to generate fluorescence, Broccoli or Spinach aptamer binding with (5Z)-5-[(3,5-difluoro-4-hydroxyphenyl)methylene]-3,5-dihydro-2,3-dimethyl-4H-imidazol-4-one, (Z)-4-(3,5-difluoro-4-hydroxybenzylidene)-1,2-dimethyl-1H-imidazol-5(4H)-one (DFHBI) to generate fluorescence, a mango aptamer, and Blue Fluorescent RNA (BFR) aptamer, but is not limited thereto.

In any one embodiment of the above-described embodiments, the reporter gene of the present invention may encode a reporter protein. In the present invention, the “reporter protein” is a marker protein that is replicated or transcribed from the sensor DNA to generate a signal capable of easily detecting production or synthesis thereof. The signal may be in various forms, such as luminescence, fluorescence, phosphorescence, color development, electron transfer, etc.

Specifically, the reporter protein may be selected from superfolder green fluorescent protein (sfGFP), green fluorescent protein (GFP), yellow fluorescent protein (YFP), red fluorescent protein (RFP), mCherry fluorescent protein, lactamase, galactosidase, horseradish peroxidase (HRP), glucose oxidase, and luciferase, but is not limited thereto. Among the above proteins, the production or synthesis of the fluorescent proteins may be identified by measuring the fluorescence of the fluorescent protein accumulated in a cell-free synthesis reaction solution. In the case of enzymes, the synthesis of the reporter protein may be identified by measuring the production level of the product using a substrate corresponding to each enzyme.

Specifically, the reporter protein may be a fluorescent protein or luciferase, and more specifically, sfGFP, firefly luciferase (FLuc), or deep sea shrimp luciferase (NLuc).

However, without being limited to the above examples, any protein or nucleic acid sequence that generates a signal capable of easily identifying the production or synthesis thereof is comprised without limitation.

As used herein, the term “target nucleic acid” refers to a nucleic acid to be detected. The target nucleic acid sequence may be DNA or RNA, and may comprise not only ssDNA and RNA, but also dsDNA sequences. dsDNA may be used by switching a complementary strand, and for example, ssDNA may be used by separating or exposing the same.

The length of the target nucleic acid sequence is not particularly limited as long as the sensor DNA of the present invention selectively expresses the reporter gene only in the presence of the target nucleic acid sequence, but the length may be, for example, 12 nt or longer. When the target nucleic acid sequence comprises the T7 promoter sequence, the length excluding the T7 promoter sequence may be 4 nt or longer, but is not limited thereto.

The composition of the present invention may further comprise any components necessary for nucleic acid and protein synthesis. Specifically, they may be components necessary for one or more reactions of cell-free replication, cell-free transcription, and cell-free protein synthesis.

As used herein, the “cell-free replication/transcription/protein synthesis” means performing, in vitro such as in a test tube, replication/transcription/protein synthesis which has been performed within cells. For example, cell-free protein synthesis means that only the components necessary for protein production, i.e., the intracellular protein synthesis machinery and factors thereof, are extracted from cells, and only the protein synthesis process is artificially repeated outside the cells in a state where the physiological control mechanism of the cells is excluded, thereby producing the target protein in a short period of time. At this time, the protein biosynthetic machinery required for cell-free protein synthesis, i.e., ribosome, initiation factor, elongation factor, terminator, aminoacyl tRNA synthetase, RNA polymerase, etc., may be used as those comprised in the cell extract, or separately added, or separately produced and used by genetic recombination technology. Similarly, cell-free replication also means performing nucleic acid replication outside cells by extracting the components necessary for nucleic acid replication from the cells.

In one embodiment, the composition of the present invention may comprise components comprised in an E. coli crude extract. In one embodiment, the composition of the present invention may comprise any component necessary for DNA repair. In one embodiment, the composition of the present invention may comprise a DNA polymerase, such as DNA polymerase I (Pol I). In one embodiment, the composition of the present invention may comprise NTP or dNTP. In one embodiment, the composition of the present invention may comprise T7 RNA polymerase. However, the components comprised in the composition of the present invention are not particularly limited as long as they selectively enable the reporter gene to be replicated/transcribed or expressed only in the presence of the target nucleic acid sequence.

In one embodiment of the present invention, the structure of the sensor DNA was designed, in which a target binding region in the form of ssDNA that anneals to a target sequence is connected to the upstream of a single strand of T7 promoter, and a reporter nucleic acid sequence (aptamer) or a protein coding sequence is connected to the downstream thereof, and it was found that the sensor DNA may selectively express the reporter nucleic acid or reporter protein only in the presence of the target sequence in an E. coli crude extract, thereby confirming that the sensor DNA may be used in detecting the target nucleic acid. In addition, when the sensor DNA is expressed in a PURE system using purified protein synthesis machinery, instead of a crude extract that already comprises DNA Pol I and dNTP necessary for DNA repair, it was found that the sensor DNA may selectively synthesize the reporter protein only in the presence of the target sequence in the presence of components to which DNA Pol I and dNTP are separately added, thereby confirming that the sensor DNA may be used in detecting the target nucleic acid.

Another aspect of the present invention provides a method of detecting a target nucleic acid, the method comprising the steps of adding a sample to the composition for detecting a target nucleic acid of the present invention; and measuring an expression level of a reporter gene by carrying out one or more reactions of cell-free replication, transcription, and protein synthesis.

The composition for detecting the target nucleic acid, the reporter gene, and the cell-free protein synthesis reaction are as described above.

In the step of measuring the expression level of the reporter gene, a method known in the art may be used. For example, when the reporter protein is a fluorescence protein or the reporter nucleic acid is a fluorescence-labeled aptamer, the expression of the reporter gene may be identified by measuring fluorescence.

In the present invention, the sample refers to a target sample comprising or not comprising a target nucleic acid to be detected, to which the method of detecting a target nucleic acid of the present invention is applied.

In one embodiment, the sample may be derived from one or more selected from the group consisting of feeds, foods, or chemical substances, but is not limited thereto.

In one embodiment, the sample may be isolated from a living organism. In one embodiment, the living organism comprises both a plant and an animal.

In one embodiment of the method of detecting a target nucleic acid of the present invention, the target nucleic acid may be a nucleic acid sequence of a virus. In this regard, the sample may be isolated from an individual suspected of being infected with the virus.

For example, the target nucleic acid may be a nucleic acid derived from a DNA or RNA virus. For example, the target nucleic acid may be a nucleic acid derived from a ssDNA or dsDNA virus.

The virus is not limited as long as its genome may be detected by the method of detecting a target nucleic acid of the present invention. For example, the virus may comprise parvovirus (e.g., human papillomavirus (HPV), polyomavirus), hepadnavirus (e.g., hepatitis B virus (HBV)); herpes virus (e.g., herpes simplex virus (HSV), varicella-zoster virus (VZV), Epstein-Barr virus (EBV), cytomegalovirus (CMV), herpes lymphotropic virus, Pityriasis rosea, Kaposi sarcoma-associated herpes virus); adenovirus (e.g., atadenovirus, abi adenovirus, ita adenovirus, mast adenovirus, siadenovirus); poxviruses (e.g., smallpox, vaccinia virus, cowpox virus, monkeypox virus, orf virus, pseudocowpox, bovine papular stomatitis virus); Tanapox virus, Yaba monkey tumor virus; molluscum contagiosum virus (MCV)); parvoviruses (e.g., adeno-associated virus (AAV), parvovirus B19, human bocavirus, bufavirus, human parv4 G1); Geminiviridae; Nanoviridae; Phycodnavirus, etc., but is not limited thereto.

### MODE FOR CARRYING OUT THE INVENTION

Hereinafter, the present invention will be described in more detail with reference to Examples and Experimental Examples. However, these Examples and Experimental Examples are only for illustrating the present invention, and thus are not intended to limit the scope of the present invention to these Examples and Experimental Examples.

### Example 1. Materials and Preparation Methods

### 1-1. Materials

ATP, GTP, UTP, CTP, creatine phosphate, creatine kinase, and E. coli MRE600 total tRNA mixture were purchased from Roche Applied Science (Indianapolis, IN, USA). L-[U-14C]leucine (11.9 GBq/mmol) was purchased from Amersham Biosciences (Uppsala, Sweden). Oligonucleotides were synthesized through Integrated DNA Technologies (Coralville, IA, USA). High-fidelity VELOCITY DNA polymerase and Phusion U Hot Start DNA polymerase were respectively purchased from Bioline (London, UK) and Thermo Fisher Scientific (Waltham, MA, USA). E. coli polymerase I, uracil-specific excision reagent (USER), and Xba1 restriction enzyme were purchased from New England Biolabs (Ipswich, MA, USA). T4 DNA ligase was purchased from Solgent Co., Ltd. (Daejeon, Korea). Other reagents were purchased from Sigma-Aldrich (St Louis, MO, USA) and used without further purification. Nano-Glo luciferase assay kit and PCR clean-up kit were purchased from Promega (Madison, WI, USA). E. coli crude extract (S12 extract) was obtained from BL21Star (DE3) using a known method.

### 1-2. Preparation of Sensor DNA

The nucleotide sequences of sfGFP, FLuc and NLuc were respectively cloned together with a 6×histidine tag at the C-terminus into NdeI and SalI restriction enzyme sites of pK7 plasmid to construct pK7-sfGFP, pK7-FLuc, and pK7-NLuc plasmids, respectively.

Information of amino acid sequences and nucleotide sequences of sfGFP, FLuc, and NLuc are shown in SEQ ID NOS: 2 to 7.

Each cloned gene was amplified by PCR using primers listed in the table below to be used as a template for a cell-free protein synthesis reaction.

TABLE 1
Synthetic oligonucleotides used in preparation of sensor DNA

| Oligomers | Sequence (5′→3′) |
|---|---|
| T7P-Broccoli | GAGCCCACACTCTACTCGACAGATACGAATATCTGGACCCGACCGTCTCCCCTAT |
| | AGTGAGTCGTATTA |
| T7P-700up- | TAGTCCTGTCGGGTTTCGC |
| Forward | |
| RBS-Reverse | CATATGTATATCTCCTTCTTAAAGTTAAACAAAATTATTTC |
| T7T-Reverse | CAAAAAACCCCTCAAGACCCG |
| 5′UTR-Forward | GACCACAACGGTTTCCCTCTAG |
| 12up-T7P-6U- | CACTGCUCAAAGUAAUACGACUCACTAUAGGGUGACCACAACGGTTTCCCTCTA |
| Forward | G |
| PV14up-T7P-6U- | GCAGCCCUGACATGUAAUACGACUCACTAUAGGGUGACCACAACGGTTTCCCT |
| Forward | CTAG |
| PV24up-T7P-8U- | ACTGUAAGAUGCAGCCCUGACAUGTAAUACGACUCACTAUAGGGUGACCACAA |
| Forward | CGGTTTCCCTCTAG |
| NLuc-NdeI- | AAAAAACATATGGTGTTCACCTTAGAA |
| Forward | |
| NLuc-SalI-Reverse | AAAAAAGTCGACTTAATGATGGTGATGGTGATGAGA |
| 5′-UTR-Xba1 | GGGAGACCACAACGGTTTCCCT |
| /5′-Phos/12up- | /5′Phos/CTAGAGGGAAACCGTTGTGGTCTCCCTATAGTGAGTCGTATTACTTTGAG |
| T7P-5′UTR-Xba1 | CAGTG |

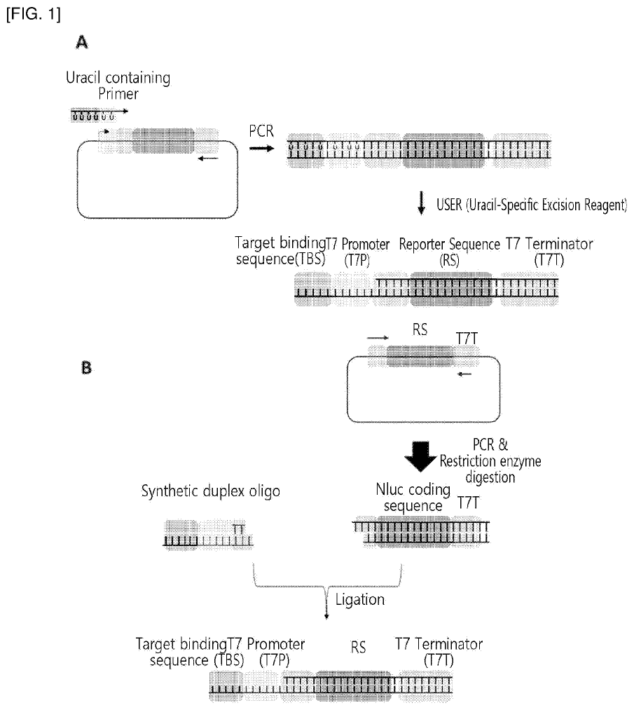

Sensor DNA having a target binding region at the end was prepared by i) excising a uracil-containing portion by a USER reaction using a uracil-containing primer or ii) chemically synthesizing an oligonucleotide containing the target binding region and a single-stranded T7 promoter sequence using a synthetic oligonucleotide, separately preparing a sequence encoding a reporter nucleic acid or protein by PCR, etc., and then ligating the same by treating with a restriction enzyme and ligase, as described in FIG. 1.

### 1) Preparation of Sensor DNA by USER Reaction

During the PCR process, a target sequence binding region sequence that recognizes the target nucleic acid was added to each gene. To prepare sensor DNA having a single-stranded target binding region sequence, a forward primer of PCR was prepared using a uracil-containing oligomer (Table 1), and the primer region was removed by treating the PCR product with the USER reaction. A first PCR reaction was performed in 100 μL of a reaction mixture containing the following composition: 2 nM of pK7-sfGFP, pK7-FLuc, or pK7-NLuc; 0.5 μM of 5′-UTR-forward and GTB-reverse primers; 1 mM of dNTP; 90 μL of 1× Velocity DNA polymerase reaction buffer and 2 units of Velocity DNA polymerase. For a second PCR reaction, the gel-extracted DNA template was amplified with uracil-containing forward oligomer/GTB-reverse oligomer pair to generate a uracil-containing DNA template strand. The PCR reaction was performed in 100 μL of a reaction mixture containing the following composition: 2 nM of linear DNA template, 0.5 μM of oligomer, 1 mM of dNTP, 1× Phusion U reaction buffer, and 2 U of Phusion U Hot Start DNA polymerase.

The USER reaction was performed at 37° C. in 100 μL of a reaction mixture containing the following composition: 160 nM of uracil-containing PCR product, 1× CutSmart buffer, and 10 U of USER enzyme. After incubation for 3 hours, the reaction mixture was passed through a PCR cleanup column to remove cleaved nucleotides. Next, the concentration of the eluted sensor DNA was then measured using a Nanodrop spectrophotometer (Thermo Fisher, Waltham, MA, USA).

### 2) Preparation of Sensor DNA Using Restriction Enzyme and Ligase

A first DNA fragment, in which an oligonucleotide (/5′-Phos/12up-T7P-5′UTR-Xba1) of 5′-terminal phosphorylated downstream sequence, consisting of the target binding region, T7 promoter, and part of the 5′-UTR, and an oligonucleotide (5′-UTR-Xba1) of the upstream sequence having a sequence complementary to the part of the 5′-UTR, were ligated, and a second DNA fragment having a reporter protein sequence treated with Xba1 restriction enzyme was ligated using ligase.

The first DNA fragment was designed to have complementary binding of the oligonucleotides of the upstream sequence and downstream sequence, and at the same time, a sticky end of the Xba1 restriction enzyme sequence, and was prepared by reacting 100 μL of a reaction mixture containing the following composition: each 45 μM of the upstream and downstream oligonucleotides; 1× annealing buffer (100 mM potassium acetate; 30 mM HEPES, pH 7.5) at 95° C. for 5 minutes and then cooling to room temperature for 20 minutes.

The PCR reaction of the second DNA fragment was performed in 100 μL of a reaction mixture containing the following composition: 0.5 μM of 5′-UTR forward primer and GTB-reverse primer; 1 mM of dNTP; 1× Velocity DNA polymerase reaction buffer; 2 units of Velocity DNA polymerase. 10 μg of the produced PCR product was incubated with 20 units of Xba1 restriction enzyme at 37° C. for 1 hour, and then passed through a PCR cleanup column to purify and produce the second DNA fragment. Ligation reaction was performed at room temperature for 1 hour in 20 μL of a reaction mixture containing the following composition: 120 μM of the first DNA fragment; 6 μM of the second DNA fragment; 400 units of T4 DNA ligase; 1×T4 DNA ligase buffer.

The sensor DNA, in which the first and second DNA fragments were ligated, was extracted using a gel extraction method, and the concentration of the eluted sensor DNA was measured using a Nanodrop spectrophotometer (Thermo Fisher, Waltham, MA, USA).

### 1-3. TASR Assay

A target nucleic acid was added to 3 μL of an annealing buffer containing 10 nM of the sensor DNA. The mixture was heated to 90° C. for 5 minutes and cooled back to room temperature to allow the target nucleic acid to anneal to an anti-target sequence, which is a target binding region of the sensor DNA. The target-annealed sensor DNA was mixed with 12 μL of a TASR assay premix solution containing the following composition:

57 mM of HEPES-KOH (pH 8.2), 1.2 mM of ATP, each 0.85 mM of CTP, GTP, and UTP, 2 mM of DL-dithiothreitol, 0.17 mg/ml of E. coli total tRNA mixture (derived from MRE600 strain), 0.64 mM of CAMP, 90 mM of potassium glutamate, 80 mM of ammonium acetate, 12 mM of magnesium acetate, 34 μg/mL of I-5-formyl-5,6,7,8-tetrahydrofolic acid (folinic acid), each 1.5 mM of 20 types of amino acids, 2% (w/v) PEG-8000, 67 mM of creatine phosphate (CP), 3.2 μg/mL of creatine kinase, and 26% (v/v) E. coli strain BL21 (DE3)-derived S12 extract.

The above reaction mixture was maintained at 30° C. for 3 hours, and then generated signals were measured by the TASR assay as described in 1-4. Meanwhile, in order to quantify the proteins synthesized during the TASR reaction, 10 μM of L-[U-¹⁴C]leucine (12.136 GBq/mmol) was added to the above reaction mixture. Subsequently, trichloroacetic acid-precipitated radioactivity was measured by a known method using a liquid scintillation counter (Wallac 1410, PerkinElmer, Waltham, MA, USA).

### 1-4. TASR Signal Analysis

10 μL of the sample of the completed assay reaction in the TASR assay using the sensor DNA with sfGFP as a reporter was mixed with 190 μL of PBS buffer, and transferred to a 96-well microplate to measure fluorescence intensity.

In experiments using FLuc as a reporter, 10 μL of the sample of the completed assay reaction was mixed with 90 μL of a luminescence buffer (20 mM Tris-HCl, pH 7.4, 1 mM magnesium carbonate, 2.7 mM magnesium sulfate, 0.1 mM EDTA, 33 mM DL-dithiothreitol, 270 M of coenzyme A, 530 μM of ATP, 470 μM of D-luciferin), and after incubation for 5 minutes at 25° C., luminescence intensity was measured.

To measure the luminescence of the synthesized NLuc by the TASR assay, all assay setups were equilibrated to room temperature, and Nano-Glo® Luciferase Assay Substrate was diluted with 50 volumes of Nano-Glo® Luciferase Assay Buffer. In a 96-well white polystyrene microplate, 10 μL of the sample of the completed assay reaction was diluted with 40 μL of PBS buffer, and supplemented with an equal volume of 2× assay buffer. After incubation at 25° C. for 5 minutes, luminescence intensity was measured using a CLARIO Star microplate reader.

The names of DNA oligomers used as target sequences for the TASR assay and information of the corresponding sequences are as shown in the following Table.

TABLE 2
Target DNA sequences used in TASR assay

| | | Tm | GC |
|---|---|---|---|
| Oligomers | Sequences (5′→3′) | (° C.) | (%) |
| T7P(00/17) | TAATACGACTCACTATAGG | 44.5 | 33.3 |
| T7P(02/17) | AGTAATACGACTCACTATAG | 47.9 | 34.8 |
| T7P(04/17) | AAAGTAATACGACTCACTATAG | 49.2 | 38.1 |
| T7P(06/17) | TCAAAGTAATACGACTCACTATAG | 49.5 | 33.3 |
| T7P(04/17) | AAAGTAATACGACTCACTATA | 45.4 | 28.6 |
| T7P(04/15) | AAAGTAATACGACTCACTA | 44.6 | 31.6 |
| T7P(04/11) | AAAGTAATACGACTC | 37.3 | 33.3 |
| T7P(08/06) | GCTCAAAGTAATACG | 39.9 | 40.0 |
| T7P(11/03) | ACTGCTCAAAGTAA | 38.8 | 35.7 |
| T7P(12/00) | CACTGCTCAAAG | 36.7 | 50.0 |
| 5′-10nts-T7P(12/00) | CGTTCTAGTACACTGCTCAAAG | — | — |
| T7P(12/00)-3′-10nts | CACTGCTCAAAGGCTACCATGG | — | — |
| 5′-10nts-T7P(12/00)- | CGTTCTAGTACACTGCTCAAAGGCTACCATGG | — | — |
| 3′-10nts | | | |
| PV-T7P(05/17) | ACATGTAATACGACTCACTATA | 48.1 | 31.8 |
| PV-T7P(12/00) | AGCCCTGACATG | 41.8 | 58.3 |
| PV-T7P(14/00) | GCAGCCCTGACATG | 49.9 | 64.4 |
| PV-T7P(17/00) | GATGCAGCCCTGACATG | 53.6 | 58.8 |
| PV-T7P(24/00) | ACTGTAAGATGCAGCCCTGACATG | 59.1 | 50.0 |
| PV(1934-1983)-50nts | TAAAGTTAAACTTTACTGTAAGATGCAGCCCTGACATGG | — | — |
| | GGTTACTAACA′ | | |

*Underlined sequence indicates the T7 promoter region.

### Example 2. Detection of Nucleic Acid Using Cell-Free Protein Synthesis

### 2-1. Synthesis of Reporter Protein Reacting to Foreign Nucleic Acid

To demonstrate the concept of generating a translation signal capable of detecting nucleic acids, a cell-free protein synthesis system initiated by a non-template strand T7 promoter sequence was designed.

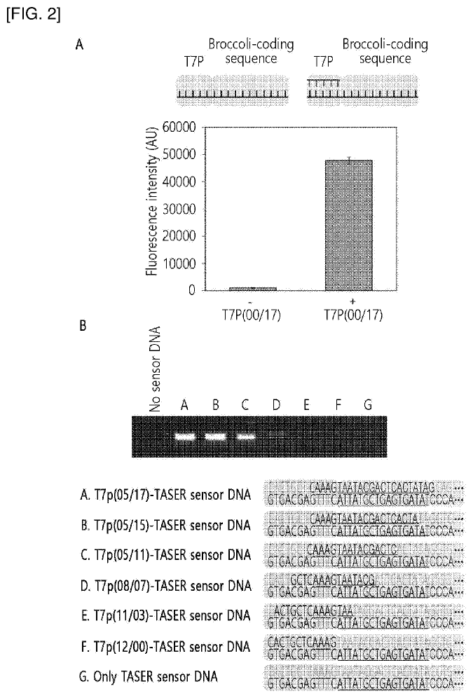

Sensor DNA was prepared by the method of FIG. 1A or FIG. 1B. When the sensor DNA prepared using the Broccoli aptamer, which is a fluorescent aptamer, as a reporter gene was directly used as a template for in vitro transcription, as expected, a significant level of transcription did not occur. However, when the oligonucleotide encoding the non-template strand of the T7 promoter sequence was annealed to sensor DNA, and in vitro transcription was performed, a significantly improved fluorescence value was obtained (FIG. 2A).

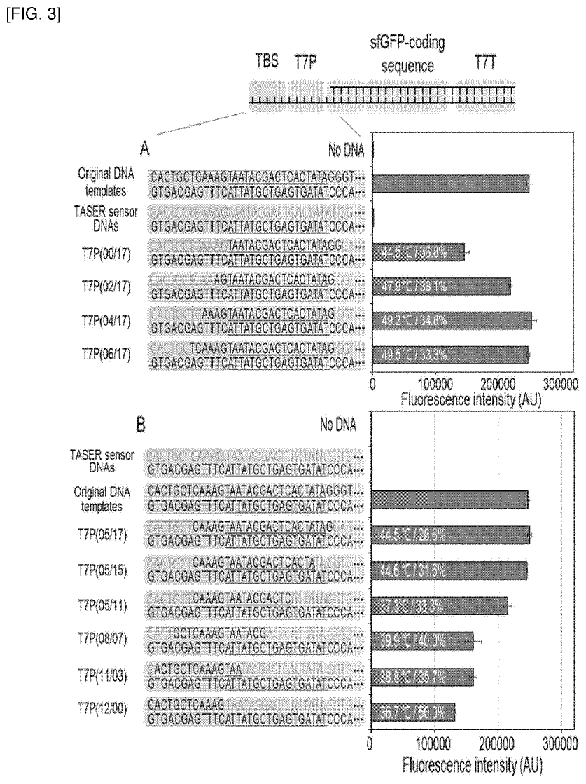

When the sensor DNA prepared using the sfGFP gene as a reporter gene was directly used as a template for cell-free protein synthesis, as expected, a significant level of sfGFP was not produced (FIGS. 3A and 3B).

However, the production amount of sfGFP was markedly enhanced by annealing the sensor DNA with the oligonucleotide encoding the non-template strand of the T7 promoter sequence (FIG. 3A). In initial experiments using an oligonucleotide synthesized exactly complementary to the T7 promoter sequence of the template strand (marked by T7P (00/17)) as a model target, sfGFP was produced with a yield of about 58.7%, as compared to a positive control reaction in which sfGFP was expressed from DNA whose entire sequence consisted of a double-stranded DNA. Furthermore, the yield of sfGFP synthesis was further enhanced when an oligonucleotide having a sequence complementary to the 5′-upstream base sequence thereof in addition to the T7 promoter region of the sensor DNA was supplied (FIG. 3A), which is most likely due to stabilization of the target-target binding site complex.

As shown in FIG. 3A, oligonucleotides with increasing numbers of additional nucleotides were investigated, and the yield of sfGFP synthesis was almost similar to the positive control reaction, when 4 or more additional base pairs were formed between the target and target binding region sequences. Under these conditions, the signal-to-noise ratio in response to 20 nM oligonucleotide was as high as 2.4×10².

These results support the fact that the transcription mechanism by T7 polymerase and T7 promoter is activated by recognizing the exogenously added nucleic acid, and through this, a sufficient amount of the reporter protein to generate a signal is produced in the cell-free protein synthesis system.

### 2-2. Target Recognition by Sensor DNA Using T7 Promoter Upstream Sequence

From the results of the above Example, it was recognized that cell-free transcription and translation reactions may be controlled by foreign nucleic acids. However, the above results were obtained by performing using the non-template sequence of the T7 promoter as a model target. In order to actually apply this to the detection of various pathogenic nucleic acids, the recognition of the target sequence by the target binding region of the sensor DNA should be programmed to occur at a separate location from the promoter sequence so that various sequences other than the fixed T7 promoter sequence may be recognized and transcription and translation may be performed therefrom. This means that target recognition should occur outside the promoter region (specifically, in the upstream sequence of the promoter so as not to affect the expression of the reporter protein encoded downstream of the promoter).

It is known that the T7 promoter must be at least partially double-stranded for the cognate T7 RNA polymerase to initiate the transcription reaction. In addition, as shown in FIG. 3A in the previous Example, since the reporter protein in the TASR assay is produced through the sequential reaction of cell-free transcription and translation, the cell-free transcription reaction must be initiated in advance for the production of the reporter protein. As a result of performing in vitro transcription after adding oligonucleotides which anneal with various lengths to the T7 promoter template strand of the sensor DNA, it was confirmed that the target-sensor duplex must comprise at least the first 15 nucleotides of the sensor DNA template strand promoter for transcription to occur (FIG. 2B).

However, the action of target-annealed sensor DNA was different in the reaction product of E. coli crude extract-based cell-free protein synthesis. When the target-probe annealing region was gradually removed from the upstream of the T7 promoter, surprisingly, sfGFP was significantly synthesized even when an oligonucleotide that did not anneal to the T7 promoter template sequence at all but only to the 5′-upstream sequence was used (T7P (12/00), FIG. 3B).

These results indicate that, even though the exact DNA synthesis pathway by the cell extract cannot be specified, the cell extract retains the ability to double-strand the T7 promoter region, in which the complementary sequence DNA is annealed to the upstream of the T7 promoter sequence of the TASR sensor DNA which is transcriptionally inactive due to the single-stranded T7 promoter and upstream sequence thereof, and the annealed DNA is used as a primer to extend the downstream thereof, thereby enabling transcription and translation of the reporter gene downstream of the promoter. Furthermore, these results imply that target nucleic acids of various sequences may be detected through cell-free transcription and translation reactions by programming the upstream of the T7 promoter of the sensor DNA with a sequence capable of annealing to the desired target sequence.

2-3. Application of E. coli Crude Extract and dNTP to TASR System

A reaction mixture for TASR analysis was composed of an E. coli crude extract comprising most of the cellular enzymes. Therefore, it was assumed that the target DNA annealed to the upstream of the T7 promoter was extended to the T7 promoter region by the action of E. coli DNA polymerase.

In particular, DNA polymerase I (Pol I), which is a DNA repair enzyme of E. coli, is assumed to be responsible for the extension of the target DNA, which was confirmed through the results of using the PURE system, instead of the crude extract-based cell-free protein synthesis system. Unlike the cell-free protein system used herein, the PURE system is a system consisting of a mixture of 36 kinds of purified enzymes and 70S ribosomes, and is prepared by separately preparing the recombinant proteins of enzymes necessary for translation and mixing the same, and therefore, the system does not comprise other cell-derived enzymes (Nature Biotechnology. 2001; 19:751-755). Therefore, RNA polymerase/NTP and DNA polymerase/dNTP were added to the cell-free transcription reaction solution, and their effects on the progression of the transcription reaction were investigated.

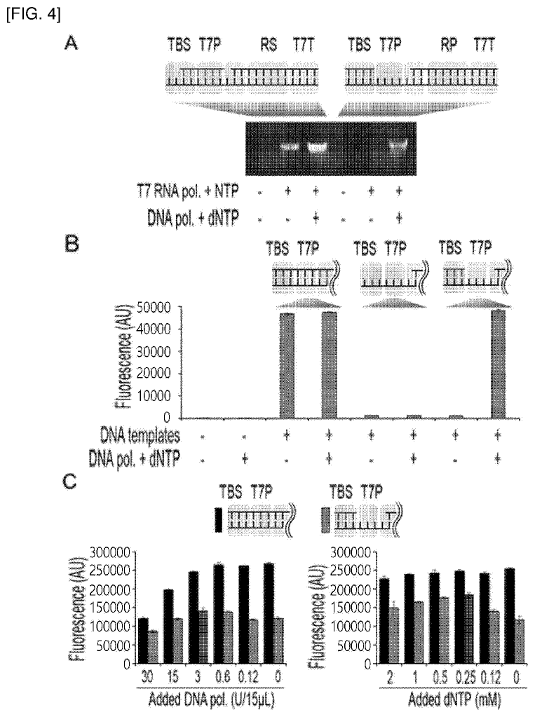

When the PURE system was used, unlike the results of the cell-free synthesis system using the crude extract, the sensor DNA annealed by T7P (12/00) could not induce mRNA synthesis through transcription. However, when Pol I and dNTP were supplemented to the reaction solution, mRNA could be generated (FIG. 4A). The results in the left of FIG. 4A showed that when a part of the target sequence was annealed with the T7 promoter, a significant level of transcription reaction occurred only by adding RNA polymerase/NTP, but as seen in the right results of FIG. 4A, when only the target binding region was annealed, transcription occurred only when DNA polymerase/dNTP was additionally added in addition to RNA polymerase/NTP. This is because the target sequence annealed at the upstream of the T7 promoter is extended by DNA polymerase, and the downstream of T7 promoter also double-helicalizes, which means that at least a part of the 5′ end of the T7 promoter must form a double helix for transcription to occur.

Pol I-mediated extension of the target annealed to the target binding region of the sensor DNA was reconfirmed by measuring the fluorescence of sfGFP synthesized from the PURE system. The results of analyzing the synthesis of sfGFP after adding DNA polymerase/dNTP to the PURE system (provided with RNA polymerase and NTPs) are shown in FIG. 4B.

In the PURE reaction mixture, the sensor DNA annealed with oligonucleotide T7P (12/00) did not produce sfGFP fluorescence, whereas the production level of sfGFP increased to a level similar to that of the control using the entire double-stranded DNA, when Pol I and dNTP were supplemented (FIG. 4B). Similar to the results of the cell-free transcription reaction in FIG. 4A, when the target sequence overlaps with a part of the T7 promoter of the sensor DNA, sfGFP protein was produced without additional addition of DNA polymerase/dNTP. However, when the target sequence anneals only with the target binding region of the sensor DNA, sfGFP synthesis occurred only when DNA polymerase/dNTP was supplemented.

Finally, the results of adding DNA polymerase/dNTP under cell-free protein synthesis conditions using the E. coli crude extract are shown in FIG. 4C. Unlike the case of using the PURE system, it was found that the reporter protein sfGFP was expressed without adding a separate DNA polymerase and dNTP, and this is because the E. coli crude extract already comprised Pol I and dNTP. However, the production of sfGFP in the reaction product using the crude extract was also rather improved by adding additional Pol I and dNTP. In this case, the fluorescence intensity increased by 50%, as compared to the original case where 3 U/mL of Pol I and 0.25 mM dNTP were supplemented to the T7P(12/00)-annealed sensor DNA template, whereas it increased slightly when only Pol I was supplemented (FIG. 4C).

Accordingly, it was confirmed that the supplementation of dNTP is an important factor in DNA duplex formation by DNA polymerase in cell-free protein synthesis.

Further, the use of E. coli crude extract-based cell-free protein synthesis system increased the degree of freedom that determines the target sequence of the nucleic acid to be detected.

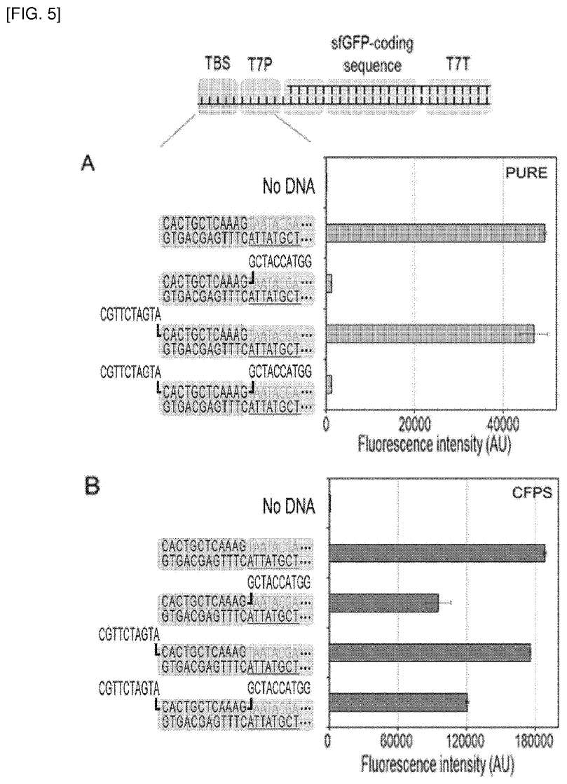

Most DNA polymerases, comprising DNA Pol I, can mediate primer elongation only when the 3′-terminus of the primer sequence is perfectly complementary to a template strand, and cannot elongate the primer when the 3′-terminus is not complementary to the template strand and thus forms a flap. When it is intended that the method of the present invention be applied to nucleic acids extracted from pathogens, the target binding region of the sensor DNA should be able to target various regions of the nucleic acid to be analyzed. When the middle region of the nucleic acid to be analyzed is targeted, the binding of the target nucleic acid to the sensor DNA inevitably forms 5′-flap and 3′-flap structures. Therefore, it was investigated whether cell-free transcription and translation reactions may proceed through binding to a target that forms a 5′-flap or a 3′-flap, and the results are shown in FIG. 5.

As shown in the results of FIG. 5A, TASR analysis was performed using the PURE system supplemented with Pol I and dNTP. By using [5′-10nts-T7P(12/00)] sequence of Table 2 as the target sequence, when the annealing of the target binding region sequence of the sensor DNA and the target sequence forms a 5′-flap, sfGFP was expressed at a level comparable to a control group that did not form flaps at both ends. However, when [T7P(12/00)-3′-10nts] sequence of Table 2 was used as the target sequence, the sensor DNA and target sequence were annealed to form a 3′-flap, and no significant level of sfGFP expression was achieved. In summary, when the PURE system was used, the presence of 5′-flap did not significantly affect sfGFP expression. However, when the 3′-flap sequence was present, the reporter protein sfGFP was not expressed at all. This was because the extension of the target sequence by Pol I was not occurred due to the 3′-flap sequence.

In contrast, in the TASR analysis using the E. coli crude extract-based cell-free protein synthesis system, about 70% of sfGFP fluorescence was observed not only in the target sequence not forming a flap and the target sequence forming a 5′-flap, but also in the presence of a 3′-flap sequence, as compared to a control group that was completely annealed without the 3′-flap sequence. This is because the flap portion was deleted by the nuclease activity present in the crude extract, and the target sequence annealed to the upstream was extended. In addition, by using the [5′-10nts-T7P(12/00)-3′-10nts] sequence of Table 2 as the target sequence, even when 5′- and 3′-flaps were simultaneously formed upon annealing with the sensor DNA, the sensor DNA could be activated in the TASR analysis using the E. coli crude extract (FIG. 5B).

These results indicate that the nuclease activity of processing the 3′-flap, which is not present in the PURE system, is comprised in the crude extract.

### 2-4. Application of Reporter Protein Nanoluciferase (NLuc) to TASR System

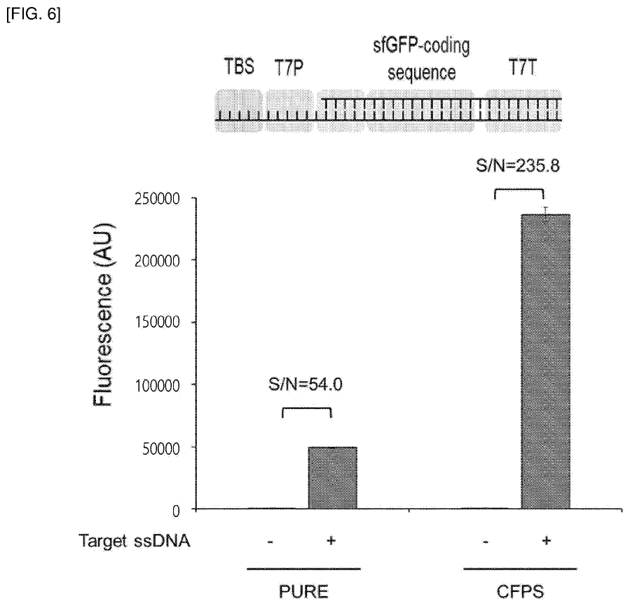

As described in the above results, both the PURE system to which DNA polymerase and dNTP were added and the crude extract-based cell-free protein synthesis system were found to be able to induce cell-free transcription and translation reactions by target sequences completely annealing to the probe region of the sensor DNA. However, in the case of the PURE system, even though DNA polymerase and dNTP were added, the signal-to-noise ratio was significantly lower than that of the reaction using the crude extract (FIG. 6:54.0 vs. 235.8). This is presumed to be because the protein productivity of the PURE system is relatively lower than that of the E. coli crude extract-based cell-free protein synthesis system.

In detail, when the same template DNA encoding sfGFP was expressed, only 30% of the sfGFP synthesized by the extract-based cell-free protein system was produced in the PURE system (FIG. 5). In addition, since the PURE system does not have a mechanism to process 3′-flap, a target sequence that forms a 3′-flap upon annealing with sensor DNA was not detected in the PURE-based TASR analysis. This means that when nucleic acid sequences such as pathogens, etc. are detected using the PURE system through TASR analysis, the sensor DNA is able to target only the 3′-terminal region of the nucleic acid to be analyzed, and TASR analysis is impossible when the middle region of the target nucleic acid is targeted, because a 3′-flap is formed. Therefore, it was concluded that it is desirable to adopt the crude extract-based cell-free protein system in order to increase the sensitivity of nucleic acid detection and to freely select and analyze the targeting region of the nucleic acid target to be detected.

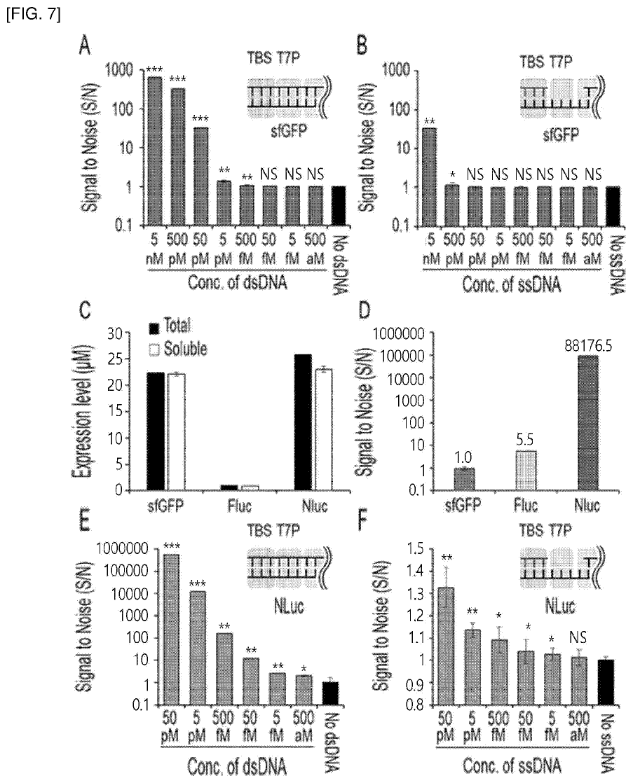

An experiment was performed to specifically identify the characteristics of TASR analysis using the E. coli crude extract-based cell-free protein synthesis system. First, a measurable DNA concentration was tested to examine the sensitivity level of the nucleic acid detection of TASR analysis. The template DNA encoding sfGFP was expressed while decreasing the concentration of template DNA in the crude extract-based cell-free protein system, and as a result, the fluorescence signal was detected until the DNA concentration was decreased to 500 fM (FIG. 7A).

The template DNA was changed to a sensor DNA detecting oligonucleotide T7P(12/00), and the oligonucleotide of the T7P(12/00) sequence was added thereto at various concentrations, and as a result, when 500 pM or more of oligonucleotide was used as the target DNA, a detectable signal was generated (FIG. 7B).

To further increase the sensitivity of TASR analysis, the reporter protein encoded by the sensor DNA was changed from sfGFP to NLuc. NLuc is a modified form of the deep sea shrimp luciferase.

The commonly used luciferase is firefly luciferase (FLuc), but NLuc was more suitable than FLuc for the E. coli extract-based cell-free protein synthesis used herein. For example, under the same reaction conditions using 10 pM of template DNA, NLuc showed the expression level and solubility yield more than 20 times higher than FLuc, respectively (FIG. 7C). In addition, the signal-to-noise ratio of cell-free synthesized NLuc was 1.5×10³ times higher than that of FLuc (FIG. 7D). Among the three proteins which were compared as reporter proteins, NLuc showed the highest expression level, solubility, and S/N value, and based on these results, NLuc was used as a reporter enzyme for TASR analysis.

The crude extract-based cell-free protein reaction was performed while decreasing the concentration of template DNA encoding NLuc. A discernible luminescence signal was detected until the DNA concentration was decreased to 500 aM (FIG. 7E). This indicates that NLuc may be detected with a sensitivity 1,000 times or more higher than the results of sfGFP expression in FIG. 7A.

When T7P (12/00) was used as the target sequence and annealed with the sensor DNA, the signal intensity was lower than that of the control group in which the entire sequence was composed of a double helix, as in the sfGFP experiment, but a significant level of luminescence signal was observed until the concentration of the target sequence reached 5 fM (FIG. 7F).

### 2-5. Detection of Parvovirus B19 Sequence Using TASR Analysis

The TASR analysis developed through the previous Example was applied to the detection of a synthetic virus DNA sequence.

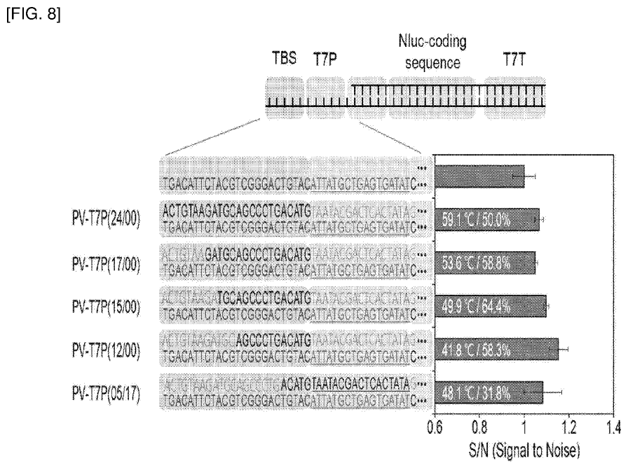

An experiment was performed to detect the Parvovirus B19 sequence which is a single-stranded DNA human virus. FIG. 8 shows the detection of the base sequence of Parvovirus B19 (PV) using NLuc as a reporter protein, and some of the base sequences of 1934-1983 of the PV genome were used as target sequences. The target binding region of the sensor DNA was designed to be complementary to the 24-base sequence [PV-T7P(24/00)] at the 3′-terminus of the PV genome 1934-1983 sequence. Then, 24 [PV-T7P(24/00)], 17 [PV-T7P(17/00)], 14 [PV-T7P(14/00)], and 12 [PV-T7P(12/00)] sequences at the 3′-terminus of the PV genome 1934-1983 were selected as target sequences, and TASR analysis was performed. In addition, the target [PV-T7P(05/17)], which anneals to 17 bases of the T7 promoter sequence in addition to 5 bases at the 3′-terminus of the PV genome 1934-1983 sequence, was also investigated. The S/N values were measured using the results in which no target sequence binding to the sensor DNA was added as a negative control. As shown in the results, a significant increase in the S/N value was observed from all target sequences that bind complementarily to the target binding region upstream of the T7 promoter sequence by 12 or more bases. It was expected that the luminescence signal would increase through more stable annealing when the Tm value of annealing was increased by increasing the length of the target sequence used, but the change in the luminescence signal according to the Tm value was not large. In other words, even when the Tm value increased to 59.1° C. through annealing with 24 bases, the change in the S/N value was not large, as compared to the case of having the Tm value of 41.8° C. through annealing with 12 bases. The increase in the luminescence signal expected when annealing a target sequence having a higher Tm value was not observed even when the Tm value increased to 59.1° C. (FIG. 8). This means that when a target sequence consisting of 12 or more base sequences is used, annealing with the sensor DNA may be stably maintained.

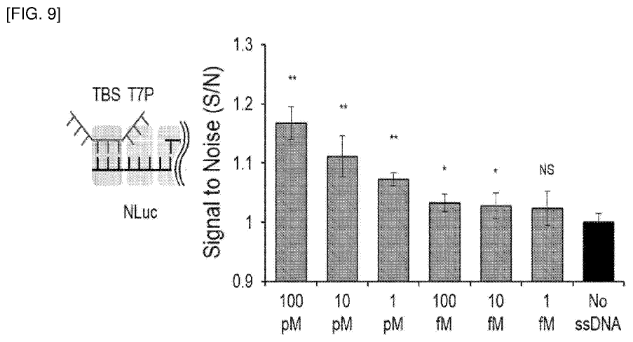

Next, PV B19 DNA (1934˜1983, PV-50nts) was tested using a sensor DNA having a target sequence binding region complementary to a target sequence consisting of 14 bases corresponding to 1958˜1971 of the synthesized PV B19 sequence, and the results are shown in FIG. 9. The target binding region of the sensor DNA was designed to consist of 14 bases complementary to the 1958-1971 base sequence of the PV genome. Since the 1934-1983 base sequence of the PV genome was used as the target sequence, the annealing of the sensor DNA and the target PV sequence forms flaps of 24 and 12 bases at the 5′ and 3′ terminals of the target sequence, respectively.

As in the previous Example, TASR analysis showed that the luminescence level increased dose-dependently with the applied PV B19 sequence.

As a result of the experiment performed by changing the concentration of PV 50nts, a statistically significant increase was observed in the luminescence level, as compared to the negative control, until the concentration of the target sequence reached 10 fM.

In other words, as in the previous result of FIG. 8, it can be seen that the E. coli crude extract-based cell-free protein synthesis system is able to detect the target even in the presence of 5′- and 3′-flaps with a sensitivity equivalent to the case without the flaps, which means that the target site of the nucleic acid to be detected may be freely set.

Based on the above description, it will be understood by those skilled in the art that the present invention may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

1. A composition for detecting a target nucleic acid, the composition comprising a sensor DNA,
wherein the sensor DNA comprises a structure of (5′) target nucleic acid sequence-binding region-promoter-reporter gene (3′),
the target nucleic acid sequence-binding region is a single-stranded DNA (ssDNA) capable of annealing to the target nucleic acid sequence, and
the promoter is a single-stranded DNA.

2. The composition of claim 1, wherein the single-stranded DNA capable of annealing to the target nucleic acid sequence has a length of 4 nt or more.

3. The composition of claim 1, wherein the target nucleic acid sequence is a DNA or an RNA.

4. The composition of claim 1, wherein the reporter gene encodes a protein selected from the group consisting of sfGFP, NLuc, and FLuc; or an aptamer.

5. The composition of claim 1, wherein the promoter is T7 promoter.

6. The composition of claim 1, further comprising components necessary for performing one or more of cell-free replication, transcription, and synthesis.

7. The composition of claim 6, comprising DNA polymerase and nucleotides.

8. The composition of claim 7, wherein the DNA polymerase is DNA polymerase I (Pol I).

9. The composition of claim 7, wherein the nucleotides are dNTP.

10. The composition of claim 1, further comprising an E. coli crude extract.

11. A method of detecting a target nucleic acid, the method comprising the steps of:
adding a sample to the composition of claim 1; and
measuring an expression level of a reporter gene by carrying out one or more reactions of cell-free replication, transcription, and protein synthesis.

12. The method of claim 11, wherein the sample is isolated from a living organism.

13. The method of claim 11, wherein the target nucleic acid is a viral DNA.


SEQUENCE LISTING (38 sequences)

SEQ ID NO: 1 - DNA, 17 bp, synthetic construct
  misc_feature 1..17: note = T7 Promoter
  source 1..17: mol_type = other DNA
taatacgactcactata

SEQ ID NO: 2 - protein, 247 aa, synthetic construct
  REGION 1..247: note = superfolderGFP (sfGFP) Amino acid sequence
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKRDHMVLHEYVNAAGITWSHPQFEKGGSHHHHHH

SEQ ID NO: 3 - DNA, 744 bp, synthetic construct
  misc_feature 1..744: note = superfolderGFP (sfGFP) Nucleotide sequence
  source 1..744: mol_type = other DNA
atgagcaaaggtgaagaactgtttaccggcgttgtgccgattctggtggaactggatggcgatgtgaacggtcacaaattcagcgtgcgtggtgaaggtgaaggcgatgccacgattggcaaactgacgctgaaatttatctgcaccaccggcaaactgccggtgccgtggccgacgctggtgaccaccctgacctatggcgttcagtgttttagtcgctatccggatcacatgaaacgtcacgatttctttaaatctgcaatgccggaaggctatgtgcaggaacgtacgattagctttaaagatgatggcaaatataaaacgcgcgccgttgtgaaatttgaaggcgataccctggtgaaccgcattgaactgaaaggcacggattttaaagaagatggcaatatcctgggccataaactggaatacaactttaatagccataatgtttatattacggcggataaacagaaaaatggcatcaaagcgaattttaccgttcgccataacgttgaagatggcagtgtgcagctggcagatcattatcagcagaataccccgattggtgatggtccggtgctgctgccggataatcattatctgagcacgcagaccgttctgtctaaagatccgaacgaaaaacgggaccacatggttctgcacgaatatgtgaatgcggcaggtattacgtggagccatccgcagttcgaaaaaggtggttctcatcaccatcaccatcactaa

SEQ ID NO: 4 - protein, 560 aa, synthetic construct
  REGION 1..560: note = Firefly luciferase (FLuc)Amino acid sequence
MVTDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVGGSHHHHHH

SEQ ID NO: 5 - DNA, 1683 bp, synthetic construct
  misc_feature 1..1683: note = Firefly luciferase (Fluc) Nucleotide sequence
  source 1..1683: mol_type = other DNA
atggtcaccgacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggggggttctcatcaccatcaccatcactaa

SEQ ID NO: 6 - protein, 221 aa, synthetic construct
  REGION 1..221: note = Nanoluciferase (NLuc) Amino acid sequence
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILANSHGFPPEVEEQAAGTLPMSCAQESGMDRHPAACASARINVGGSHHHHHH

SEQ ID NO: 7 - DNA, 666 bp, synthetic construct
  misc_feature 1..666: note = Nanoluciferase (NLuc) Nucleotide sequence
  source 1..666: mol_type = other DNA
atggtgttcaccttagaagactttgtcggtgactggcgccagaccgccggttacaacctggaccaagtcctggagcagggtggggtttcatctctgttccagaacctgggtgttagtgtgaccccgatccagcgcattgtactgtccggcgaaaacggcctgaagatcgatattcacgtgatcatcccgtatgaaggcctgtctggagaccaaatgggtcagatcgagaaaatcttcaaagttgtttacccggtggatgaccaccactttaaagttatcctgcactacggcactctggtcatcgacggcgtgactccgaacatgatcgactacttcggccgtccgtacgaaggtattgctgtttttgatggtaagaagatcaccgttactggcaccctgtggaacggtaataaaatcatcgacgaacgcctgattaacccggacggctccctgctgttccgtgtgaccattaacggcgttaccggctggcgtctgtgcgaacgtatcctggcaaacagccacggttttccgccggaagttgaagaacaagcggctggcactctccctatgtcctgcgcgcaggagtccggcatggaccgccacccggcagcctgcgcttccgcacgcattaacgttggtggttctcatcaccatcaccatcattaa

SEQ ID NO: 8 - DNA, 69 bp, synthetic construct
  misc_feature 1..69: note = T7P-Broccoli
  source 1..69: mol_type = other DNA
gagcccacactctactcgacagatacgaatatctggacccgaccgtctcccctatagtgagtcgtatta

SEQ ID NO: 9 - DNA, 19 bp, synthetic construct
  misc_feature 1..19: note = T7P-700up-Forward
  source 1..19: mol_type = other DNA
tagtcctgtcgggtttcgc

SEQ ID NO: 10 - DNA, 41 bp, synthetic construct
  misc_feature 1..41: note = RBS-Reverse
  source 1..41: mol_type = other DNA
catatgtatatctccttcttaaagttaaacaaaattatttc

SEQ ID NO: 11 - DNA, 21 bp, synthetic construct
  misc_feature 1..21: note = T7T-Reverse
  source 1..21: mol_type = other DNA
caaaaaacccctcaagacccg

SEQ ID NO: 12 - DNA, 22 bp, synthetic construct
  misc_feature 1..22: note = 5'UTR-Forward
  source 1..22: mol_type = other DNA
gaccacaacggtttccctctag

SEQ ID NO: 13 - DNA, 55 bp, synthetic construct
  source 1..55: mol_type = other DNA
  modified_base 7: mod_base = OTHER; note = uracil
  modified_base 13: mod_base = OTHER; note = uracil
  modified_base 16: mod_base = OTHER; note = uracil
  modified_base 22: mod_base = OTHER; note = uracil
  modified_base 28: mod_base = OTHER; note = uracil
  modified_base 33: mod_base = OTHER; note = uracil
cactgctcaaagtaatacgactcactatagggtgaccacaacggtttccctctag

SEQ ID NO: 14 - DNA, 57 bp, synthetic construct
  source 1..57: mol_type = other DNA
  modified_base 8: mod_base = OTHER; note = uracil
  modified_base 15: mod_base = OTHER; note = uracil
  modified_base 18: mod_base = OTHER; note = uracil
  modified_base 24: mod_base = OTHER; note = uracil
  modified_base 30: mod_base = OTHER; note = uracil
  modified_base 35: mod_base = OTHER; note = uracil
gcagccctgacatgtaatacgactcactatagggtgaccacaacggtttccctctag

SEQ ID NO: 15 - DNA, 67 bp, synthetic construct
  source 1..67: mol_type = other DNA
  modified_base 5: mod_base = OTHER; note = uracil
  modified_base 10: mod_base = OTHER; note = uracil
  modified_base 18: mod_base = OTHER; note = uracil
  modified_base 23: mod_base = OTHER; note = uracil
  modified_base 28: mod_base = OTHER; note = uracil
  modified_base 34: mod_base = OTHER; note = uracil
  modified_base 40: mod_base = OTHER; note = uracil
  modified_base 45: mod_base = OTHER; note = uracil
actgtaagatgcagccctgacatgtaatacgactcactatagggtgaccacaacggtttccctctag

SEQ ID NO: 16 - DNA, 27 bp, synthetic construct
  misc_feature 1..27: note = NLuc-NdeI-Forward
  source 1..27: mol_type = other DNA
aaaaaacatatggtgttcaccttagaa

SEQ ID NO: 17 - DNA, 36 bp, synthetic construct
  misc_feature 1..36: note = NLuc-SalI-Reverse
  source 1..36: mol_type = other DNA
aaaaaagtcgacttaatgatggtgatggtgatgaga

SEQ ID NO: 18 - DNA, 22 bp, synthetic construct
  misc_feature 1..22: note = 5'-UTR-Xba1
  source 1..22: mol_type = other DNA
gggagaccacaacggtttccct

SEQ ID NO: 19 - DNA, 55 bp, synthetic construct
  misc_feature 1..55: note = /5'-Phos/ 12up-T7P-5'UTR-Xba1
  source 1..55: mol_type = other DNA
ctagagggaaaccgttgtggtctccctatagtgagtcgtattactttgagcagtg

SEQ ID NO: 20 - DNA, 19 bp, synthetic construct
  misc_feature 1..19: note = T7P(00/17)
  source 1..19: mol_type = other DNA
taatacgactcactatagg

SEQ ID NO: 21 - DNA, 20 bp, synthetic construct
  misc_feature 1..20: note = T7P(02/17)
  source 1..20: mol_type = other DNA
agtaatacgactcactatag

SEQ ID NO: 22 - DNA, 22 bp, synthetic construct
  misc_feature 1..22: note = T7P(04/17)
  source 1..22: mol_type = other DNA
aaagtaatacgactcactatag

SEQ ID NO: 23 - DNA, 24 bp, synthetic construct
  misc_feature 1..24: note = T7P(06/17)
  source 1..24: mol_type = other DNA
tcaaagtaatacgactcactatag

SEQ ID NO: 24 - DNA, 21 bp, synthetic construct
  misc_feature 1..21: note = T7P(04/17)
  source 1..21: mol_type = other DNA
aaagtaatacgactcactata

SEQ ID NO: 25 - DNA, 19 bp, synthetic construct
  misc_feature 1..19: note = T7 Promoter
  source 1..19: mol_type = other DNA
aaagtaatacgactcacta

SEQ ID NO: 26 - DNA, 15 bp, synthetic construct
  misc_feature 1..15: note = T7P(04/11)
  source 1..15: mol_type = other DNA
aaagtaatacgactc

SEQ ID NO: 27 - DNA, 15 bp, synthetic construct
  misc_feature 1..15: note = T7P(08/06)
  source 1..15: mol_type = other DNA
gctcaaagtaatacg

SEQ ID NO: 28 - DNA, 14 bp, synthetic construct
  misc_feature 1..14: note = T7P(11/03)
  source 1..14: mol_type = other DNA
actgctcaaagtaa

SEQ ID NO: 29 - DNA, 12 bp, synthetic construct
  misc_feature 1..12: note = T7P(12/00)
  source 1..12: mol_type = other DNA
cactgctcaaag

SEQ ID NO: 30 - DNA, 22 bp, synthetic construct
  misc_feature 1..22: note = 5'-10nts-T7P(12/00)
  source 1..22: mol_type = other DNA
cgttctagtacactgctcaaag

SEQ ID NO: 31 - DNA, 22 bp, synthetic construct
  misc_feature 1..22: note = T7P(12/00)-3'-10nts
  source 1..22: mol_type = other DNA
cactgctcaaaggctaccatgg

SEQ ID NO: 32 - DNA, 32 bp, synthetic construct
  misc_feature 1..32: note = 5'-10nts-T7P(12/00)-3'-10nts
  source 1..32: mol_type = other DNA
cgttctagtacactgctcaaaggctaccatgg

SEQ ID NO: 33 - DNA, 22 bp, synthetic construct
  misc_feature 1..22: note = PV-T7P(05/17)
  source 1..22: mol_type = other DNA
acatgtaatacgactcactata

SEQ ID NO: 34 - DNA, 12 bp, synthetic construct
  misc_feature 1..12: note = PV-T7P(12/00)
  source 1..12: mol_type = other DNA
agccctgacatg

SEQ ID NO: 35 - DNA, 14 bp, synthetic construct
  misc_feature 1..14: note = PV-T7P(14/00)
  source 1..14: mol_type = other DNA
gcagccctgacatg

SEQ ID NO: 36 - DNA, 17 bp, synthetic construct
  misc_feature 1..17: note = PV-T7P(17/00)
  source 1..17: mol_type = other DNA
gatgcagccctgacatg

SEQ ID NO: 37 - DNA, 24 bp, synthetic construct
  misc_feature 1..24: note = PV-T7P(24/00)
  source 1..24: mol_type = other DNA
actgtaagatgcagccctgacatg

SEQ ID NO: 38 - DNA, 50 bp, synthetic construct
  misc_feature 1..50: note = PV(1934-1983)-50nts
  source 1..50: mol_type = other DNA
taaagttaaactttactgtaagatgcagccctgacatggggttactaaca